(19) United States
(12) Patent Application Publication
(10) Pub. No.: US 2012/0022515 A1
(43) Pub. Date: Jan. 26, 2012

(54) HEMOSTATIC APPLICATOR AND HEMOSTATIC MODULE

(75) Inventors: Gwo-Bin Lee, Tainan City (TW); Xi-Zhang Lin, Tainan City (TW); Chong-Jeh Lo, Pingtung County (TW); Sheng-Chieh Huang, Tainan City (TW); Yi-Yuan Chang, Pingtung County (TW)

(73) Assignee: INDUSTRIAL TECHNOLOGY RESEARCH INSTITUTE, Hsinchu (TW)

(21) Appl. No.: 12/891,767

(22) Filed: Sep. 27, 2010

(30) Foreign Application Priority Data
Jul. 22, 2010 (TW) 99124180

(51) Int. Cl. A61B 18/04

(52) U.S. Cl. 606/28

(57) ABSTRACT

A hemostatic applicator adapted to stop a bleeding site from bleeding is provided. The hemostatic applicator includes a magnetic part, an anti-adhesion layer and a non-magnetic part. The magnetic part is suitable to be heated to a temperature with a high frequency electromagnetic field. The anti-adhesion layer is formed on a surface of the magnetic part, and the magnetic part contacts the bleeding site through the anti-adhesion layer. The non-magnetic part is connected with the magnetic part. A hemostatic module is also provided.

### CROSS-REFERENCE TO RELATED APPLICATION

This application claims the priority benefit of Taiwan application serial no. 99124180, filed on Jul. 22, 2010. The entirety of the above-mentioned patent application is hereby incorporated by reference herein and made a part of this specification.

### BACKGROUND OF THE DISCLOSURE

1. Field of the Invention

The disclosure relates to a hemostatic applicator and a hemostatic module including the hemostatic applicator. More particularly, the disclosure relates to a hemostatic applicator which stops bleeding with a high frequency electromagnetic field and a hemostatic module including the hemostatic applicator.

2. Description of Related Art

With the advancement in society and technology, humans have higher probabilities of being exposed to hazardous environment or being plagued with sickness. In addition, case histories or deaths involving internal tissue or visceral organ hemorrhage resulted from accidents or diseases occupy a large proportion.

Currently, hemostatic methods focusing on mass hemorrhage can be divided into physical methods and chemical methods in general. In most of physical hemostatic methods, blood vessels of visceral organs under mass hemorrhage are tied with surgical sutures or embolized with medications, so that the tissues lack blood and stop bleeding. Although physical methods can rapidly stop bleeding, the vessel embolism involved therein requires complicated surgical processes and may lead to necrosis of normal tissues due to ischemia so as to result in various complications. In chemical hemostatic methods, chemical medications such as haemostatic powder, hemostat, and histoacryl are applied or sprayed on tissues under mass hemorrhage to stop bleeding. However, as chemical medications cannot stably adhere on tissues under mass hemorrhage, the hemorrhage cannot be stopped effectively and the time for saving patients is delayed. Furthermore, when the visceral organ is under mass hemorrhage which cannot be stopped by conventional hemostatic methods, surgeons may decide to remove the entire visceral organ. This method not only makes the recovery process difficult and painful for the patient, but the patient also loses many physiologies, which leads to severe sequela.

Accordingly, conventional hemostatic methods cannot stop bleeding rapidly, simply, effectively, and safely; thus, a new hemostatic applicator is demanded.

### SUMMARY OF THE INVENTION

A hemostatic applicator and a hemostatic module are introduced herein to stop bleeding rapidly, simply, effectively, and safely.

A hemostatic applicator adapted to stop a bleeding site from bleeding is introduced herein. The hemostatic applicator includes a magnetic part, an anti-adhesion layer, and a non-magnetic part. The magnetic part is suitable to be heated to a temperature with a high frequency electromagnetic field. The anti-adhesion layer is formed on a surface of the magnetic part. Herein, the magnetic part contacts the bleeding site through the anti-adhesion layer. The non-magnetic part is connected with the magnetic part.

A hemostatic module including a high frequency generating apparatus and a hemostatic applicator is further introduced herein. The high frequency generating apparatus includes a coil that is driven by a current to generate a high frequency electromagnetic field. The hemostatic applicator is adapted to stop a bleeding site from bleeding. The hemostatic applicator includes a magnetic part, an anti-adhesion layer, and a non-magnetic part. The magnetic part is suitable to be heated to a temperature with the high frequency electromagnetic field. The anti-adhesion layer is formed on a surface of the magnetic part. Herein, the magnetic part contacts the bleeding site through the anti-adhesion layer. The non-magnetic part is connected with the magnetic part.

In light of the foregoing, the hemostatic applicator in the disclosure sinters the bleeding site with the magnetic part heated by the high frequency generating apparatus to stop the bleeding. Specifically, the anti-adhesion layer is formed on the surface of the magnetic part, such that the bleeding site and blood are prevented from adhering to the magnetic part to avoid the possible secondary damage of the bleeding site caused by the adhesion. Therefore, the hemostatic applicator can rapidly, simply, effectively, and safely stop the bleeding.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a schematic diagram illustrating a hemostatic module according to the disclosure.

FIG. 2 is a schematic diagram illustrating a hemostatic applicator according to an exemplary embodiment.

FIG. 3 is a schematic diagram illustrating a hemostatic applicator according to an exemplary embodiment.

FIG. 4 is a schematic diagram illustrating a hemostatic applicator according to an exemplary embodiment.

FIG. 5 is a schematic diagram illustrating a hemostatic applicator according to an exemplary embodiment.

### DESCRIPTION OF EMBODIMENTS

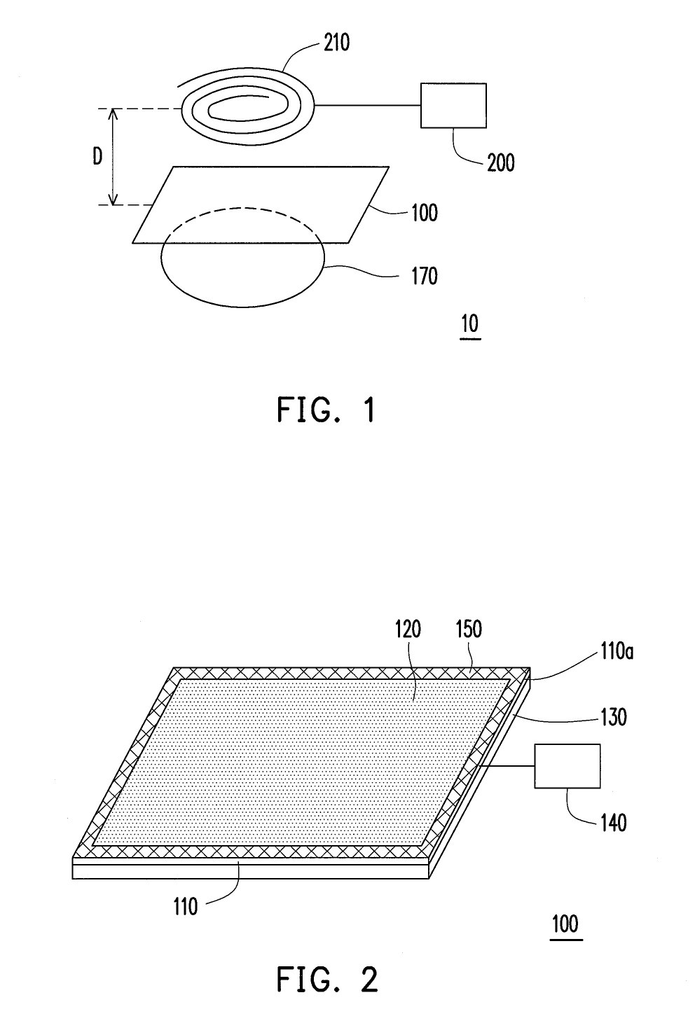

FIG. 1 is a schematic diagram illustrating a hemostatic module according to the disclosure. FIG. 2 is a schematic diagram illustrating a hemostatic applicator according to an exemplary embodiment. Referring to FIGS. 1 and 2 simultaneously, the present exemplary embodiment provides a hemostatic module 10 which includes a high frequency generating apparatus 200 and a hemostatic applicator 100. The hemostatic applicator 100 is suitable for stopping a bleeding site 170 from bleeding. The hemostatic applicator 100 includes a magnetic part 110, an anti-adhesion layer 120, and a non-magnetic part 130. The magnetic part 110 is suitable to be heated to a temperature with a high frequency electromagnetic field. The high frequency electromagnetic field is generated by the high frequency generating apparatus 200. In details, the high frequency generating apparatus 200, for example, includes a coil 210. The coil 210 is driven by a current to generate a high frequency alternative electromagnetic field capable of heating the magnetic part 110. In the high frequency alternative electromagnetic field generated by the high frequency generating apparatus 200, the magnetic part 110 generates an eddy current and hysteresis due to the high frequency alternative electromagnetic field. Consequently, a high temperature is generated to sinter the bleeding site 170 for stopping the bleeding site 170 from bleeding. Here, a distance D between the magnetic part 110 and the coil 210 of the high frequency generating apparatus 200 ranges from 0 centimeter (cm) to 20 cm, for example. A frequency of the high frequency electromagnetic field, for instance, ranges from 1 kiloHertz (kHz) to 500 kHz. A time for heating the magnetic part 110 by the high frequency generating apparatus 200 ranges, for example, from 3 second (s) to 300 s. A temperature of the magnetic part 110 after the heating ranges, for example, from 80° C. to 250° C. In an exemplary embodiment, the magnetic part 110 is capable of reaching a high temperature of 100° C. to 120° C. in 5 s to 10 s, so that the bleeding site 170 is sintered and stops bleeding. The magnetic part 110 is fabricated by a magnetic material, such as stainless steel and so on, that can generate heat under a magnetic field effect.

The anti-adhesion layer 120 is formed on a surface 110a of the magnetic part 110. Herein, the magnetic part 110 contacts the bleeding site 170 through the anti-adhesion layer 120. The anti-adhesion layer 120 is configured to prevent the tissue and blood of the bleeding site 170 from adhering to the magnetic part 110 due to high temperature. Thus, the bleeding site 170 is prevented from being damaged when removing the hemostatic applicator 100. The anti-adhesion layer 120 is fabricated by, for example, Teflon. Alternatively, by processing on the magnetic part 110, the anti-adhesion layer 120 is formed on the surface 110a of the magnetic part 110, for example.

The non-magnetic part 130 is connected to the magnetic part 110. The non-magnetic part 130 is a part that is not affected by the high frequency magnetic field, such that normal tissues or the operator are prevented from being damaged by high temperature. The non-magnetic part 130 is fabricated with a material that does not generate heat in a varying magnetic field. This material is a non-magnetic material such as ceramics, engineering plastic, heat resistant tape, and biocompatible gel. Herein, the engineering plastic is fabricated by, for example, polyetheretherkotone (PEEK), Teflon, and other materials. The heat resistant tape is fabricated with Teflon, for instance.

In the present exemplary embodiment, the hemostatic applicator 100 further includes a temperature sensor 140 connected to the magnetic part 110. The temperature sensor 140 is, for example, connected to the magnetic part 110 and the high frequency generating apparatus 200. The temperature sensor 140 outputs a sensing signal to the high frequency generating apparatus 200 according to a temperature change of the magnetic part 110. In other words, the sensing signal output by the temperature sensor 140 performs a feedback control to the high frequency generating apparatus 200. As a consequence, the high frequency generating apparatus 200 heats the magnetic part 110 to a suitable temperature to prevent the temperature of the magnetic part 110 from being too high such that the bleeding site 170 is damaged, or from being too low so that the bleeding is not stopped.

A method of stopping the bleeding site 170 from bleeding with the hemostatic applicator 100 in the present exemplary embodiment includes the following. The hemostatic applicator 100 with the suitable size is chosen according to the location, area, and condition of the bleeding site 170. The hemostatic applicator 100 is adhered to the bleeding site 170 through adhesion. Alternatively, the hemostatic applicator 100 is held by the operator to contact the bleeding site 170. Then, the coil 210 of the high frequency generating apparatus 200 approaches the hemostatic applicator 100, such that the magnetic part 110 of the hemostatic applicator 100 is heated by a high frequency magnetic field generated by the high frequency generating apparatus 200. Here, the heated magnetic part 110 contacts the bleeding site 170 through the anti-adhesion layer 120 to sinter the bleeding site 170 and stops the bleeding. Moreover, the temperature sensor 140 is utilized for detection the temperature of the heated magnetic part 110 so as to control the temperature within a suitable range and control the suitable time for heating. After the hemostasis process, the hemostatic applicator 100 is removed and subsequent medical procedures such as suturing are performed. It should be noted that since the magnetic part 110 contacts the bleeding site 170 through the anti-adhesion layer 120, the tissue and blood of the bleeding site 170 are prevented from adhering to the magnetic part 110 due to high temperature, such that the damaging of the bleeding site 170 is avoided when the hemostatic applicator 100 is removed. Further, the high frequency generating apparatus 200 is any apparatus capable of generating a suitable high frequency electromagnetic field, and is not limited in the disclosure.

Several exemplary embodiments of the hemostatic applicator in the disclosure are described in the following sequentially to further illustrate components and methods of using the hemostatic applicator. It should be noted that in the exemplary embodiments below, the components in the hemostatic applicator are similar to those aforementioned. Thus, materials and functions of the components can also referred to the above. The following focuses on the illustration of connection methods, shapes, and differences of the components. It should be illustrated that the components of the hemostatic applicator can also have other sizes, shapes, and connection methods, and the invention is not limited thereto.

Referring to FIG. 2, in the present exemplary embodiment, the hemostatic applicator 100 is a hemostatic patch, for example, suitable for stopping the bleeding site 170 from bleeding. Moreover, the hemostatic applicator 100 is, for example, suitable to be adhered to the bleeding site 170 with a large range and imprecise hemorrhagic spots. In details, the hemostatic applicator 100 is suitable to be heated by the high frequency generating apparatus 200 and thus configured to stop the bleeding site 170 from bleeding. The hemostatic applicator 100 includes the magnetic part 110, the anti-adhesion layer 120 formed on the surface 110a of the magnetic part 110, an adhesion part 150 formed on the surface 110a of the magnetic part 110, and the non-magnetic part 130. The magnetic part 110 is a stainless steel sheet with a thickness ranging from 0.01 millimeter (mm) to 1 mm, for example. The non-magnetic part 130 is a Teflon sheet or Teflon tape with heat resistance (i.e. with resistance of 250° C.), for instance. The magnetic part 110 is adhered on the non-magnetic part 130. In the present exemplary embodiment, the hemostatic applicator 110 can have various shapes corresponding to the bleeding site 170. For example, the hemostatic applicator 110 can be cut into a suitable size and shape depending on the bleeding site 170.

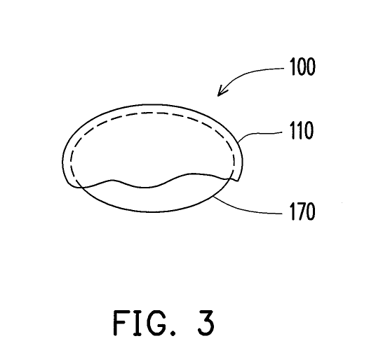

In the present embodiment, the adhesion part 150 is located in the periphery of the anti-adhesion layer 120, for example. The adhesion part 150 is configured to adhere to the external periphery of the bleeding site 170. In other words, the hemostatic applicator 100 contacts the bleeding site 170 through the anti-adhesion layer 120. Additionally, the hemostatic applicator 100 is fixed on the bleeding site 170 through the adhesion part 150, so that the magnetic part 110 can stop the bleeding. Herein, the adhesion part 150 is fabricated with silicone, Teflon, and biocompatible gel. In addition, in another exemplary embodiment, as shown in FIG. 3, the magnetic part 110 of the hemostatic applicator 100 has flexibility, for instance. The magnetic part 110 can thus cover the bleeding site 170. This hemostatic applicator 100 is suitable for (but not limited to) a non-planar bleeding site 170, such as truncated limbs or visceral organs with imprecise hemorrhagic spots. It should be noted that for better illustration, FIG. 3 merely shows the magnetic part 110 and omits the remaining components of the hemostatic applicator 100.

In the present exemplary embodiment, since the hemostatic applicator 100 is easily adhered to the bleeding site 170 with a large range, the hemostatic applicator 100 is suitable for (but not limited to) the bleeding site 170 on a surface of tissues or visceral organs under mass hemorrhage where the hemorrhagic spot can not be determined. Hence, the hemostatic applicator 100 can rapidly sinter the hemorrhagic spot to stop the bleeding so as to prevent the time delay when saving patients. Moreover, the hemostatic applicator 100 can be further cut according to the shape and area of the bleeding site 170. The magnetic part 110 is fixed on the bleeding site 170 through the adhesion part 150. As a result, the bleeding can be stopped effectively and the normal tissues in the periphery of the bleeding site 170 are prevented from being damaged. It should be noted that since the magnetic part 110 contacts the bleeding site 170 through the anti-adhesion layer 120, the tissue and blood of the bleeding site 170 are prevented from adhering to the magnetic part 110 due to high temperature, such that the damaging of the bleeding site 170 is avoided when the hemostatic applicator 100 is removed. Therefore, the hemostatic applicator in the present exemplary embodiment can rapidly, simply, effectively, and safely stop the bleeding.

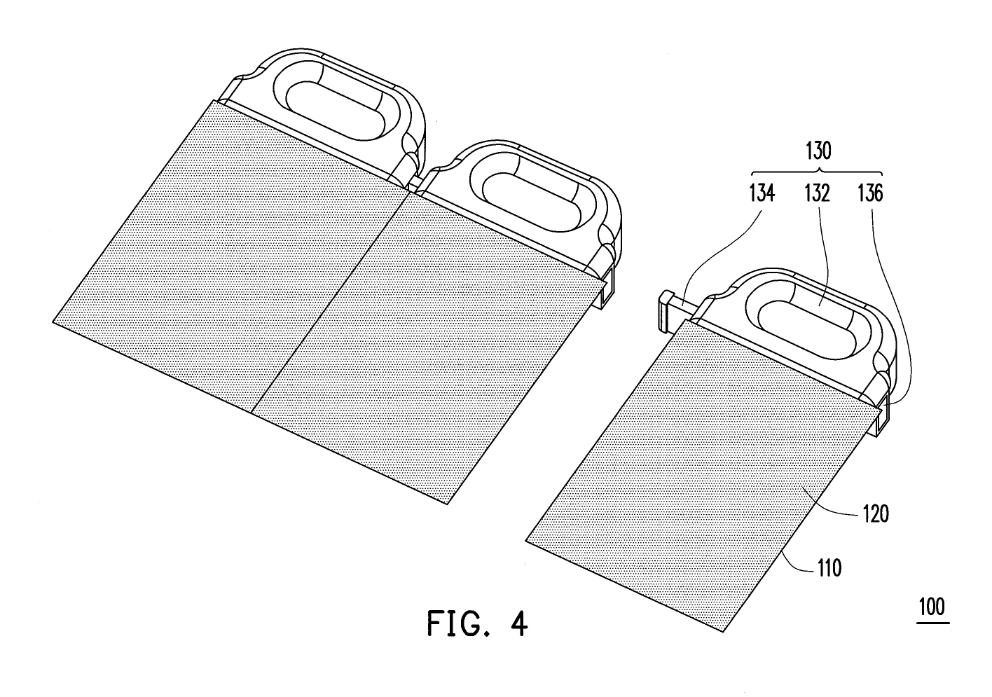

FIG. 4 is a schematic diagram illustrating a hemostatic applicator according to an exemplary embodiment. Referring to FIGS. 1 and 4 simultaneously, in the present exemplary embodiment, the hemostatic applicator 100 is, for instance, an assembled hemostatic applicator suitable for stopping the bleeding site 170 from bleeding, and is, for instance, suitable for (but not limited to) cross-sectional hemorrhage resulted from the surgical removal of partial visceral organs. Specifically, the hemostatic applicator 100 is suitable to be heated by the high frequency generating apparatus 200 and thus configured to stop the bleeding site 170 from bleeding. The hemostatic applicator 100 includes the magnetic part 110, the anti-adhesion layer 120, the non-magnetic part 130, and the temperature sensor 140 connected to the magnetic part 110 (not shown in FIG. 4, please refer to FIG. 2). The magnetic part 110 is, for example, a stainless steel sheet suitable to be heated to a temperature with a high frequency electromagnetic field. The high frequency electromagnetic field is generated by the high frequency generating apparatus 200. The magnetic part 110 contacts the bleeding site 170 through the anti-adhesion layer 120. The anti-adhesion layer 120 is fabricated by, for example, Teflon or ceramics. By processing on the magnetic part 110, the anti-adhesion layer 120 is formed on the surface of the magnetic part 110.

In the present exemplary embodiment, the non-magnetic part 130 is, for example, a handheld base fabricated by heat resistant Teflon (i.e. with heat resistance up to 250° C.), for example. The non-magnetic part 130 includes a handheld part 132, a tenon 134, and a groove 136. The tenon 134 and the groove 136 are located at respective sides of the handheld part 132. Therefore, a plurality of hemostatic applicators 100 can be assembled to or detached from one another through the tenon 134 and the groove 136. In other words, in the present exemplary embodiment, the operator can assemble the hemostatic applicator 100 according to the location or area of the bleeding site 170 so as to optimize the hemostasis. It should be noted that although a plurality of hemostatic applicators 100 in the present exemplary embodiment is assembled or detached through the tenon 134 and the groove 136, persons with common knowledge in the art should understand that the hemostatic applicators 100 can also be connected through connection components other than the tenon 134 and the groove 136.

In the present exemplary embodiment, as the operator can assemble the hemostatic applicator 100 according to the location or area of the bleeding site 170, the hemostatic applicator is thus suitable for (but not limited to) cross-sectional hemorrhage resulted from partial damage or surgical removal of partial visceral organs. Hence, the hemostatic applicator 100 can attain superior hemostatic efficiency within a short period of time. The bleeding at the cross-section is stopped rapidly to prevent the time delay for saving patients. In addition, as the non-magnetic part 130 of the hemostatic applicator 100 has the handheld part 132, the operator can adjust the position of the magnetic part 110 directly according to the bleeding condition of the bleeding site 170. Consequently, the bleeding can be stopped effectively and the normal tissues in the periphery of the bleeding site 170 are prevented from being damaged by the magnetic part 110. It should be noted that since the magnetic part 110 contacts the bleeding site 170 through the anti-adhesion layer 120, the tissue and blood of the bleeding site 170 are prevented from adhering to the magnetic part 110 due to high temperature, such that the damaging of the bleeding site 170 is avoided when the hemostatic applicator 100 is removed. Therefore, the hemostatic applicator in the present exemplary embodiment can rapidly, simply, effectively, and safely stop the bleeding.

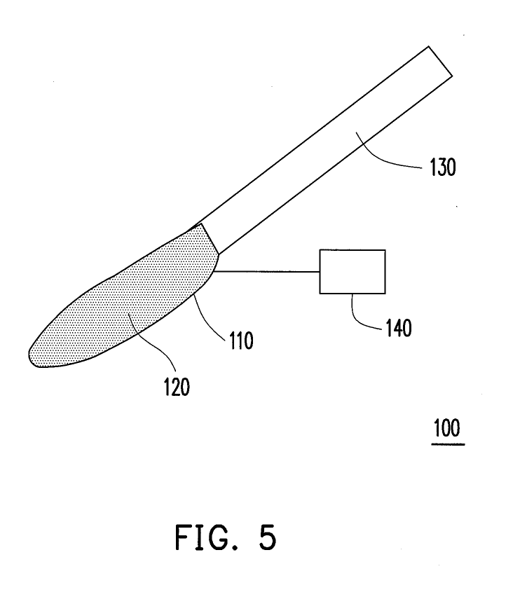

FIG. 5 is a schematic diagram illustrating a hemostatic applicator according to an exemplary embodiment. Referring to FIGS. 1 and 5 simultaneously, in the present exemplary embodiment, the hemostatic applicator 100 is a handheld hemostatic applicator having high applicability and mobility, for example. The hemostatic applicator 100 is suitable for stopping the bleeding site 170 from bleeding and is suitable for (but not limited to) multiple bleeding sites 170 randomly distributed in the visceral organs or tissues, or multiple bleeding sites 170 that cannot be processed simultaneously, for example. Specifically, the hemostatic applicator 100 is suitable to be heated by the high frequency generating apparatus 200 and thus configured to stop the bleeding site 170 from bleeding. The hemostatic applicator 100 includes the magnetic part 110, the anti-adhesion layer 120, the non-magnetic part 130, and the temperature sensor 140 connected to the magnetic part 110. The magnetic part 110 is, for example, a stainless steel sheet or a cutting blade. The magnetic part 110 is suitable to be heated to a temperature with the high frequency electromagnetic field. The high frequency electromagnetic field is generated by the high frequency generating apparatus 200. The magnetic part 110 contacts the bleeding site 170 through the anti-adhesion layer 120. The anti-adhesion layer 120 is fabricated by, for example, Teflon. By processing on the magnetic part 110, the anti-adhesion layer 120 is formed on the surface of the magnetic part 110, for example.

In the present exemplary embodiment, the non-magnetic part 130 is, for example, a long handle fabricated by heat resistant Teflon (i.e. with heat resistance up to 250° C.), for example. The magnetic part 110 is connected to one end of the long handle of the non-magnetic part 130, for instance. The magnetic part 110 and the non-magnetic part 130 are detachably connected through the tenon, for instance. In other words, the hemostatic applicator 100 has a configuration similar to a surgical blade, and is substantially used for cutting and stopping the bleeding simultaneously, for example. Further, since the magnetic part 110 and the non-magnetic part 130 are detachably connected, the used magnetic part 110 can be replaced by another un-used magnetic part 110. It should be illustrated that although in the present exemplary embodiment, the magnetic part 110 and the non-magnetic part 130 are connected through the tenon (not shown), persons with common knowledge in the art should understand that the magnetic part 110 and the non-magnetic part 130 of the hemostatic applicator 100 can also be connected through other connection components other than the tenon.

In the present exemplary embodiment, the hemostatic applicator 100 has high applicability and mobility. The hemostatic applicator 100 is, for example, suitable for (but not limited to) multiple bleeding sites 170 randomly distributed in visceral organs or tissues, or multiple bleeding sites 170 that cannot be processed simultaneously. As a consequence, the hemostatic applicator 100 is removed repeatedly to stop the bleeding immediately and rapidly. Moreover, normal tissues other than the bleeding sites 170 are prevented from being damaged by the magnetic part 110. It should be noted that since the magnetic part 110 contacts the bleeding site 170 through the anti-adhesion layer 120, the tissue and blood of the bleeding site 170 are prevented from adhering to the magnetic part 110 due to high temperature, such that the damaging of the bleeding site 170 is avoided when the hemostatic applicator 100 is removed. Therefore, the hemostatic applicator can rapidly, simply, effectively, and safely stop the bleeding.

In summary, the hemostatic applicator in the disclosure sinters the bleeding site with the magnetic part heated by the high frequency generating apparatus to stop the bleeding. More specifically, the anti-adhesion layer is formed on the surface of the magnetic part, such that the bleeding site and blood are prevented from adhering to the magnetic part to avoid the possible secondary damage of the bleeding site caused by adhesion. In addition, the hemostatic applicator of the disclosure can be in patches, assembled applicators, handheld applicators, and so on. Thus, the suitable hemostatic applicator can be chosen depending on the location, range and condition of the bleeding site to stop the bleeding rapidly and safely. In other words, the hemostatic applicator of the disclosure can greatly reduce the time for hemostasis so as to increase hemostatic efficiency and prevent normal tissues from being damaged to avoid complications after surgery. Therefore, the hemostatic applicator can rapidly, simply, effectively, and safely stop the bleeding.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A hemostatic applicator adapted to stop a bleeding site from bleeding, the hemostatic applicator comprising:
a magnetic part, suitable to be heated to a temperature with a high frequency electromagnetic field;
an anti-adhesion layer, formed on a surface of the magnetic part, wherein the magnetic part contacts the bleeding site through the anti-adhesion layer; and
a non-magnetic part, connected with the magnetic part.

2. The hemostatic applicator as claimed in claim 1, wherein a frequency of the high frequency electromagnetic field ranges from 1 kHz to 500 kHz.

3. The hemostatic applicator as claimed in claim 1, wherein the temperature ranges from 80° C. to 250° C.

4. The hemostatic applicator as claimed in claim 1, wherein a time for heating the magnetic part ranges from 3 second (s) to 300 s.

5. The hemostatic applicator as claimed in claim 1, wherein the magnetic part comprises stainless steel.

6. The hemostatic applicator as claimed in claim 1, wherein the non-magnetic part comprises ceramics, engineering plastic, heat resistant tape, and biocompatible gel.

7. The hemostatic applicator as claimed in claim 1, wherein the anti-adhesion layer comprises Teflon or ceramic material.

8. The hemostatic applicator as claimed in claim 1, further comprising a temperature sensor connected to the magnetic part.

9. The hemostatic applicator as claimed in claim 1, wherein a thickness of the magnetic part ranges from 0.01 millimeter (mm) to 1 mm.

10. The hemostatic applicator as claimed in claim 1, wherein the magnetic part has a sheet shape.

11. The hemostatic applicator as claimed in claim 1, wherein the magnetic part has flexibility.

12. The hemostatic applicator as claimed in claim 1, further comprising an adhesion part formed on the surface of the magnetic part and configured for adhering to an external periphery of the bleeding site.

13. The hemostatic applicator as claimed in claim 12, wherein the adhesion part is located in a periphery of the anti-adhesion layer.

14. The hemostatic applicator as claimed in claim 12, wherein the adhesion part comprises silicone, Teflon, and biocompatible gel.

15. The hemostatic applicator as claimed in claim 1, wherein the non-magnetic part comprises a hand-held part.

16. The hemostatic applicator as claimed in claim 15, wherein the non-magnetic part comprises a tenon and a groove located at respective sides of the hand-held part.

17. The hemostatic applicator as claimed in claim 1, wherein the non-magnetic part is a long handle, and the magnetic part is connected to one end of the long handle.

18. The hemostatic applicator as claimed in claim 17, wherein the magnetic part is a cutting blade.

19. The hemostatic applicator as claimed in claim 1, wherein the magnetic part and the non-magnetic part are connected detachably.

20. A hemostatic module, comprising:
a high frequency generating apparatus, comprising a coil to be driven by a current to generate a high frequency electromagnetic field; and
a hemostatic applicator, adapted to stop a bleeding site from bleeding, and comprising:
a magnetic part, suitable to be heated to a temperature with the high frequency electromagnetic field;
an anti-adhesion layer, formed on a surface of the magnetic part, wherein the magnetic part contacts the bleeding site through the anti-adhesion layer; and
a non-magnetic part, connected with the magnetic part.

21. The hemostatic module as claimed in claim 20, wherein a distance between the magnetic part and the coil ranges from 0 centimeter (cm) to 20 cm.

22. The hemostatic module as claimed in claim 20, wherein the hemostatic applicator further comprises a temperature sensor, the temperature sensor is connected to the magnetic part and the high frequency generating apparatus, and outputs a sensing signal to the high frequency generating apparatus according to a temperature change of the magnetic part.
